(19) United States
(12) Patent Application Publication
(10) Pub. No.: US 2022/0105361 A1
(43) Pub. Date: Apr. 7, 2022

(54) USE OF PHOTOBIOMODULATION THERAPY TO TREAT INFLAMMATORY AND/OR METABOLIC CONDITIONS

(71) Applicant: SYMBYX PTY LTD, Sydney, New South Wales (AU)

(72) Inventor: Brian Bicknell, Freshwater (AU)

(21) Appl. No.: 17/421,818

(22) PCT Filed: Jan. 10, 2020
(86) PCT No.: PCT/AU2020/050015

§ 371 Date: Jul. 9, 2021

(30) Foreign Application Priority Data
Jan. 11, 2019 (AU) 2019900083
May 17, 2019 (AU) 2019901677

(51) Int. Cl. A61N 5/06; A61N 5/067

(52) U.S. Cl. CPC A61N 5/0624; A61N 5/067; A61N 2005/0645; A61N 2005/0651; A61N 2005/0659

(57) ABSTRACT

Disclosed is a method for altering the microbiome of a subject, and for preventing or treating an inflammatory condition and/or a metabolic condition in a subject, the method comprising treating the subject with light at a wavelength which 5 causes an alteration in the microbiome of the subject, and a device for use in the method.

### TECHNICAL FIELD

The present disclosure relates to a method of altering the microbiome of a subject, and to a method of treating or preventing an inflammatory condition and/or a metabolic condition, such as metabolic syndrome, a neurological condition or a cardiovascular condition.

### BACKGROUND

There is a well established link between the microbiome and a number of inflammatory and metabolic conditions, such as metabolic syndrome (including type 2 diabetes, and obesity), neurological disorders and cardiovascular disease. Metabolic syndrome such as type 2 diabetes, neurological disorders such as Parkinson's disease and Alzheimer's disease, and cardiovascular diseases such as atherosclerosis are significant health issues in many countries throughout the world. For example, a neurological disorder such as Parkinson's disease (PD) alone affects over 1 million people in the United States.

Parkinson's disease is a progressive disorder of the central nervous system which is caused by the degeneration of the pigmented neurons in the Substantia nigra of the brain, resulting in decreased dopamine availability to the striatum. Clinically, the disease is characterized by a decrease in spontaneous movements, gait difficulty, postural instability, rigidity and tremor.

The most common and effective drug for treatment of Parkinson's disease is levodopa, either administered alone or in combination with a peripheral dopa decarboxylase inhibitor such as carbidopa. However, the treatment loses its effectiveness after a prolonged period of use, and patients often exhibit abnormal motor side effects such as dyskinesias and dystonias. These problems limit the long-term benefit that can be achieved with this drug.

Metabolic syndrome is a cluster of conditions that can occur together, and that can result in increased risk of heart disease, stroke and type 2 diabetes. The conditions associated with metabolic syndrome include high blood pressure, high blood sugar (insulin resistance), high cholesterol, excess body fat around the waist (central obesity), high LDL, high serum triglycerides, and low HDL. Treatment for metabolic syndrome include diet, exercise and drugs which treat individual conditions of the syndrome.

What is needed is an alternative treatment for inflammatory conditions and/or a metabolic conditions, such as metabolic syndrome, neurological disorders such as Parkinson's disease, and cardiovascular disease.

### SUMMARY

The inventor has found that the microbiome of a subject can be altered by treating the subject with light in the infra-red wavelength, such as photobiomodulation therapy.

The inventor has reasoned that altering the microbiome of subjects suffering from, or at risk of suffering from, a metabolic condition (such as metabolic syndrome, or a cardiovascular condition (such as atherosclerosis)) and/or an inflammatory condition (such as a neurological condition (e.g., Parkinson's disease)), to enhance the relative proportion of beneficial microbes, may result in a reduction or amelioration of the symptoms of the condition.

The inventor has shown that an inflammatory condition and/or a metabolic condition in a subject can be treated by irradiating the subject with light, such as photobiomodulation therapy, at a wavelength which causes an alteration in the microbiome of the subject.

Accordingly, a first aspect provides a method of preventing or treating an inflammatory condition and/or a metabolic condition in a subject, comprising treating the subject with light, typically with photobiomodulation therapy (PBMT), at a wavelength which causes an alteration in the microbiome of the subject.

An alternative first aspect provides a light, typically photobiomodulation light, for use in a method of preventing or treating a metabolic condition and/or inflammatory condition in a subject, wherein the light is at a wavelength which causes an alteration in the microbiome of the subject.

A second aspect provides a method of preventing or treating a metabolic condition and/or a neurological condition and/or a cardiovascular condition, in a subject, comprising treating the subject with light, typically with photobiomodulation therapy (PBMT), at a wavelength which causes an alteration in the microbiome of the subject.

An alternative second aspect provides a light, typically photobiomodulation light, for use in a method of preventing or treating a metabolic condition and/or a neurological condition and/or a cardiovascular condition in a subject, wherein the light is at a wavelength which causes an alteration in the microbiome of the subject.

A third aspect provides a method for altering the microbiome of a subject, comprising treating the subject with light, typically photobiomodulation therapy (PBMT), at a wavelength which causes an alteration in the microbiome of the subject.

An alternative third aspect provides a light, typically photobiomodulation light, for use in a method of altering the microbiome of a subject, wherein the light is at a wavelength which causes an alteration in the microbiome of the subject.

A fourth aspect provides a method of preventing or treating Parkinson's disease in a subject, comprising treating the subject with light, typically photobiomodulation therapy (PBMT), at a wavelength which causes an alteration in the microbiome of the subject.

An alternative fourth aspect provides a light, typically photobiomodulation light, for use in a method of preventing or treating Parkinson's disease in a subject, wherein the light is at a wavelength which causes an alteration in the microbiome of the subject.

A fifth aspect provides a light emitting device when used for preventing or treating an inflammatory condition and/or a metabolic condition in a subject, wherein the device is arranged to administer light, typically photobiomodulation light, to the patient at a wavelength and location which causes an alteration in the microbiome of the subject.

A sixth aspect provides a light emitting device when used for preventing or treating a metabolic condition and/or a neurological condition and/or a cardiovascular condition in a subject, wherein the device is arranged to administer light, typically photobiomodulation light, to the patient at a wavelength and location which causes an alteration in the microbiome of the subject.

A seventh aspect provides a light emitting device when used for preventing or treating a neurological condition in a subject, wherein the device is arranged to administer light, typically photobiomodulation light, to the patient at a wavelength and location which causes an alteration in the microbiome of the subject.

An eighth aspect provides a light emitting device when used for altering a microbiome of a subject, wherein the device is arranged to administer light, typically photobiomodulation light, to the patient at a wavelength and location which causes an alteration in the microbiome of the subject.

A ninth aspect provides a method of reducing body weight in a subject, comprising treating the subject with light, typically with photobiomodulation therapy (PBMT), at a wavelength which causes an alteration in the microbiome of the subject.

An alternative ninth aspect provides light, typically photobiomodulation light, for use in a method of reducing body weight in a subject, wherein the light is at a wavelength which causes an alteration in the microbiome of the subject.

A tenth aspect provides a method of reducing cholesterol levels, and/or reducing LDL levels, and/or reducing serum triglyceride levels, in a subject, comprising treating the subject with light, typically with photobiomodulation therapy (PBMT), at a wavelength which causes an alteration in the microbiome of the subject.

An alternative tenth aspect provides light, typically photobiomodulation light, for use in a method of reducing cholesterol levels and/or reducing LDL levels, and/or reducing serum triglyceride levels, in a subject, wherein the light is at a wavelength which causes an alteration in the microbiome of the subject.

An eleventh aspect provides a method of reducing the effects of ageing in a subject, comprising treating the subject with light, typically with photobiomodulation therapy (PBMT), at a wavelength which causes an alteration in the microbiome of the subject.

An alternative eleventh aspect provides light, typically photobiomodulation light, for use in a method of reducing the effects of ageing in a subject, wherein the light is at a wavelength which causes an alteration in the microbiome of the subject.

A twelfth aspect provides a method of increasing resilience in a subject, comprising treating the subject with light, typically with photobiomodulation therapy (PBMT), at a wavelength which causes an alteration in the microbiome of the subject.

An alternative twelfth aspect provides light, typically photobiomodulation light, for use in a method of increasing resilience in a subject, wherein the light is at a wavelength which causes an alteration in the microbiome of the subject.

A thirteenth aspect provides a light emitting device for use in preventing or treating an inflammatory condition and/or a metabolic condition in a subject, wherein the device is structured to administer light, typically photobiomodulation light, to the patient at a wavelength and location which causes an alteration in the microbiome of the subject.

A fourteenth aspect provides a light emitting device for use in preventing or treating a metabolic condition and/or a neurological condition and/or a cardiovascular condition in a subject, wherein the device is structured to administer light, typically photobiomodulation light, to the patient at a wavelength and location which causes an alteration in the microbiome of the subject.

A fifteenth aspect provides a light emitting device for use in preventing or treating a neurological condition in a subject, wherein the device is structured to administer light, typically photobiomodulation light, to the patient at a wavelength and location which causes an alteration in the microbiome of the subject.

A sixteenth aspect provides a light emitting device for use in altering a microbiome of a subject, wherein the device is structured to administer light, typically photobiomodulation light, to the patient at a wavelength and location which causes an alteration in the microbiome of the subject.

A seventeenth aspect provides a light emitting device for use in reducing body weight, and/or reducing cholesterol levels, and/or reducing LDL levels, and/or reducing serum triglyceride levels, and/or reducing the effects of ageing, and/or increasing resilience, in a subject, wherein the device is structured to administer light, typically photobiomodulation light, to the patient at a wavelength and location which causes an alteration in the microbiome of the subject.

An eighteenth aspect provides a light emitting device when used for reducing body weight, and/or reducing cholesterol levels, and/or reducing LDL levels, and/or reducing serum triglyceride levels, and/or reducing the effects of ageing, and/or increasing resilience, in a subject, wherein the device is structured to administer light, typically photobiomodulation light, to the patient at a wavelength and location which causes an alteration in the microbiome of the subject.

### BRIEF DESCRIPTION OF THE DRAWINGS

FIG. 1 shows a front perspective of an inner layer of an embodiment of a light emitting device described herein.

FIG. 2 shows a rear perspective of a middle layer of an embodiment of a light emitting device described herein.

FIG. 3 shows a front perspective of the outer layer of an embodiment of a light emitting device described herein.

FIG. 4 shows a side view of an example of a light housing for the middle layer referred to in FIG. 2.

FIG. 5 is a diagram further illustrating the arrangement of the light housing in FIG. 2, with front view (A) and side view (B).

FIG. 6 is a diagram showing an example of the distribution of points of irradiation on the abdomen of a subject.

### DETAILED DESCRIPTION

As described herein, the inventor has found that applying light in the infrared wavelength, more typically the near infrared wavelength, to the abdomen of subjects results in an alteration of the microbiome. As described herein, by applying to the abdomen of healthy mice low level laser light in the infrared wavelength, the microbiome composition of the mice was changed, and in particular the relative proportion of at least one genus of beneficial mouse bacteria (Allobaculum sp.) was increased. Allobaculum sp. is known to be associated with a healthy mouse microbiome and is reduced in unhealthy mouse microbiomes.

The inventor has also found that by applying low level laser light in the infrared wavelength, more typically the near-infrared wavelength, to the abdomen of healthy humans, the microbiome composition of humans was changed, and in particular the relative proportion of beneficial bacteria (for example, Akkermansia sp., Bifidobacterium sp., Lachnospira sp., Alistipes sp., Bacteroides sp., Coprococcus sp., Viellonella sp., Faecalibacterium sp., Roseburia sp., Dialister sp., Sutterella sp., and RF39) was increased. Additionally, the relative proportion of some unhealthy bacteria (e.g., Dorea sp., Blautia sp., Bilophila sp., Trabulsiella sp., Turicibacter sp., Collinsella tercoris, Holdmania, Clostridium symbiosum, Eggerthella sp., Ruminococcus and Paraeggerthella hongkongensis) was decreased.

Accordingly, in one aspect, there is provided a method of altering the microbiome of a subject, comprising treating the subject with light, typically photobiomodulation therapy, at a wavelength and amount effective to cause an alteration in the microbiome of the subject.

In one embodiment, the alteration in the microbiome of the subject comprises increasing the proportion of at least one beneficial bacterial phylum, genus or species in the microbiome of the subject. As used herein, a beneficial bacterial phylum, genus or species is a bacterial phylum, genus or species which is associated with a healthy microbiome. In some embodiments in which the subject is a dog, rat or mouse, the beneficial bacterial phylum, or species may be Allobaculum sp.

In various embodiments in which the subject is a human, the beneficial bacterial phylum, genus or species may comprise one or more bacterial genus or species selected from the group consisting of:
(a) Akkermansia sp., Bifidobacterium sp., Lachnospira sp., Alistipes sp., Bacteroides sp., Coprococcus sp., Viellonella sp., Faecalibacterium sp., Roseburia sp., Dialister sp., Sutterella sp., Methanobrevibacter sp., and RF39;
(b) Lactobacillus sp., Bifidobacteria sp., Akkermansia sp., Faecalibacterium sp., Roseburia sp. and Bacteroides sp.
(c) Akkermansia sp., Lachnospira sp., Alistipes sp., Viellonella sp., Faecalibacterium sp., Roseburia sp., Dialister sp., Sutterella sp., Parabacteroides sp., and Prevotella sp.;
(d) Akkermansia sp., Bifidobacterium sp., Lachnospira sp., Alistipes sp., Coprococcus sp., Viellonella sp., Faecalibacterium sp., Roseburia sp., Dialister sp., Sutterella sp., Methanobrevibacter sp., and RF39; or
(e) Akkermansia sp., Bifidobacterium sp., Faecalibacterium sp., and Roseburia sp.;
(f) Akkermansia mucinophila, Bifidobacterium spp., Faecalibacterium prausnitzii and Roseburia spp.;
(g) Akkermansia mucinophila;
(h) Faecalibacterium prausnitzii;
(i) Bifidobacterium spp.;
(j) Roseburia sp.;
(k) Agathobacter sp.;
(l) Eubacterium sp.; or
(m) Ruminococcus sp.

In one embodiment, the beneficial bacteria are selected from the group consisting of Akkermansia mucinophila, Bifidobacterium longum., Faecalibacterium prausnitzii. and Roseburia inulinivorans.

In one embodiment, the beneficial bacterial phylum, genus or species is Akkermansia mucinophila.

In one embodiment, the alteration in the microbiome comprises a decrease in the ratio of the proportion of bacteria of the phylum Firmicutes to the proportion of bacteria of the phylum Bacteroidetes in the microbiome.

An increase in the proportion of a bacterial phylum, genus or species in the microbiome of a subject is an increase in the number or abundance of that phylum, genus or species in the microbiome after treatment relative to the number or abundance of the phylum, genus or species in the microbiome before treatment. A decrease in the proportion of a bacterial phylum, genus or species in the microbiome is a decrease in the number or abundance of that phylum, genus or species in the microbiome after treatment relative to the number or abundance of the phylum, genus or species in the microbiome before treatment.

A decrease in the ratio of the proportion of bacteria of the phylum Firmicutes to the proportion of bacteria of the phylum Bacteroidetes in the microbiome is a decrease in the ratio in the microbiome after treatment relative to the ratio in the microbiome before treatment.

Typically, the alteration in the microbiome is determined from a sample of the microbiome. In some embodiments, the increase or decrease in the proportion of a bacterial phylum, genus or species in the microbiome, or the decrease in the ratio of the proportion of bacteria of the phylum Firmicutes to the proportion of bacteria of the phylum Bacteroidetes in the microbiome, is determined from a representative sample of the microbiome, typically obtained before and after treatment. A sample of the microbiome is typically a faecal sample.

In some embodiments, the number or abundance of a phylum, genus or species in a sample is expressed as a percentage of the total number of bacteria in that sample. Therefore, in some embodiments, the increase or decrease in the proportion of a bacterial phylum, genus or species in a microbiome following treatment is an increase or decrease in the percentage of that bacterial phylum, genus or species in a sample of the microbiome expressed as a percentage of the total number of bacteria in that sample of the microbiome relative to the percentage of that bacterial phylum, genus or species in a sample of the microbiome before treatment. In some embodiments, the proportion of a bacterial phylum, genus or species in a microbiome is the number of that bacterial phylum, genus or species in a sample of the microbiome relative to the number of a reference phylum, genus or species in the sample.

In one embodiment, the alteration in the microbiome of the subject comprises decreasing the proportion of at least one unhealthy bacterial phylum, genus or species in the microbiome of the subject. For example, in mice, examples of an unhealthy bacteria include species of Burkholderales, Clostridium, Shuttleworthia and Nocardioides. In humans, examples of unhealthy bacteria include Collinsella tercoris, Holdinania, Clostridium symbiosum, Eggerthella and Paraeggerthella hongkongensis.

In one embodiment, the alteration in the microbiome comprises a decrease in the ratio of the proportion of bacteria of the phylum Firmicutes to the proportion of bacteria of the phylum Bacteroidetes. In various embodiments, the decrease in the ratio of the proportion of bacteria of the phylum Firmicutes to the proportion of bacteria of the phylum Bacteroidetes is a decrease of at least 10%, 20%, 30%, 40%, 50%, 60%, or 70% in the ratio.

One aspect provides a method of altering the microbiome of a subject, comprising administering to the subject an effective amount of light at a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm). In one embodiment, the wavelength is 808 nm.

The microbiome is now known to have a direct relationship to many metabolic, neurological and cardiovascular diseases such as metabolic syndrome, type 2 diabetes, atherosclerosis, Parkinson's disease and Alzheimer's disease.

Accordingly, one aspect provides a method of preventing or treating a metabolic condition (such as, for example, obesity, metabolic syndrome, high cholesterol, high LDL, high serum triglycerides, or a cardiovascular condition), and/or an inflammatory condition (such as a neurological condition), in a subject, comprising treating the subject with light, typically by photobiomodulation therapy (PBMT), at a wavelength which causes an alteration in the microbiome of the subject. In one embodiment, the alteration in the microbiome of the subject comprises an increase in the proportion of at least one beneficial bacterial genus or species in the microbiome of the subject. In some embodiments in which the subject is a dog, rat or mouse, the beneficial bacterial phylum, genus or species may be Allobaculum sp. In various embodiments in which the subject is a human, the beneficial bacterial phylum, genus or species may comprise one or more bacterial genus or species selected from the group consisting of:
(a) Akkermansia sp., Bifidobacterium sp., Lachnospira sp., Alistipes sp., Bacteroides sp., Coprococcus sp., Viellonella sp., Faecalibacterium sp., Roseburia sp., Dialister sp., Sutterella sp., Methanobrevibacter sp., and RF39;
(b) Lactobacillus sp., Bifidobacteria sp., Akkermansia sp., Faecalibacterium sp., Roseburia sp. and Bacteroides sp.
(c) Akkermansia sp., Lachnospira sp., Alistipes sp., Viellonella sp., Faecalibacterium sp., Roseburia sp., Dialister sp., Sutterella sp., Parabacteroides sp., and Prevotella sp.;
(d) Akkermansia sp., Bifidobacterium sp., Lachnospira sp., Alistipes sp., Coprococcus sp., Viellonella sp., Faecalibacterium sp., Roseburia sp., Dialister sp., Sutterella sp., Methanobrevibacter sp., and RF39; or
(e) Akkermansia sp., Bifidobacterium sp., Faecalibacterium sp., and Roseburia sp.;
(f) Akkermansia mucinophila, Bifidobacterium spp., Faecalibacterium prausnitzii and Roseburia spp.;
(g) Akkermansia mucinophila;
(h) Faecalibacterium prausnitzii;
(i) Bifidobacterium spp.;
(j) Roseburia spp.;
(k) Agathobacter sp.;
(l) Eubacterium sp.; or
(m) Ruminococcus sp.

In one embodiment, the beneficial bacteria are selected from the group consisting of Akkermansia mucinophila, Bifidobacterium longum., Faecalibacterium prausnitzii. and Roseburia inulinivorans.

In one embodiment, the beneficial bacterial phylum, genus or species is Akkermansia mucinophila.

In one embodiment, the alteration in the microbiome comprises a decrease in the ratio of the proportion of bacteria of the phylum Firmicutes to the proportion of bacteria of the phylum Bacteroidetes. In various embodiments, the decrease in the ratio of the proportion of bacteria of the phylum Firmicutes to the proportion of bacteria of the phylum Bacteroidetes is a decrease of at least 10%, 20%, 30%, 40%, 50%, 60% or 70% in the ratio.

In one embodiment, the alteration in the microbiome of the subject comprises decreasing the proportion of at least one unhealthy bacterial phylum, genus or species in the microbiome of the subject. For example, in mice, examples of an unhealthy bacteria include species of Burkholderales, Clostridium, Shuttleworthia and Nocardioides. In humans, examples of unhealthy bacteria include Collinsella tercoris, Holdmania, Clostridium symbiosum, Eggerthella and Paraeggerthella hongkongensis.

In one embodiment, the alteration in the microbiome of the subject comprises increasing the proportion of at least one beneficial bacterial phylum, genus or species, and decreasing the proportion of at least one unhealthy, bacterial phylum, genus or species, in the microbiome of the subject.

In one embodiment, the metabolic condition is one or more metabolic disorders selected from the group consisting of Type 2 diabetes, obesity, high blood pressure, high blood triglycerides, high cholesterol, high LDL, and low levels of HDL cholesterol. In one embodiment, the metabolic condition is Type 2 diabetes. In various embodiments, the metabolic condition is:
(a) high cholesterol;
(b) high cholesterol, high serum triglycerides and high LDL;
(c) high cholesterol, high serum triglycerides, high LDL and obesity;
(d) high LDL;
(e) high serum triglycerides;
(f) high cholesterol and obesity;
(g) high LDL and obesity;
(h) high serum triglycerides and obesity; or
(i) obesity.

As described in the Examples, the inventor has found that applying low level laser light in the infrared wavelength, typically the near infrared wavelength, to the abdomen of humans suffering from elevated cholesterol levels results in a reduction in the cholesterol level, a reduction in body weight, a reduction in LDL and a reduction in serum triglyceride levels.

Accordingly, there is provided a method of reducing body weight and/or reducing cholesterol levels and/or reducing LDL levels and/or reducing serum triglyceride levels in a subject, comprising treating the subject with light, at a wavelength and amount effective to cause an alteration in the microbiome of the subject. Typically, the subject is treated with photobiomodulation therapy (PBMT) at a wavelength and amount effective to cause an alteration in the microbiome of the subject.

One aspect provides a method of treating or preventing metabolic syndrome in a subject, comprising administering to the subject an effective amount of light at a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

One aspect provides a method of reducing body weight in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

One embodiment provides a method of reducing cholesterol levels in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

One embodiment provides a method of reducing LDL levels in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

One embodiment provides a method of reducing serum triglyceride levels in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

One aspect provides a method of treating or preventing a cardiovascular condition in a subject, comprising treating the subject with light, at a wavelength and amount effective to cause an alteration in the microbiome of the subject. Typically, the subject is treated with photobiomodulation therapy (PBMT) at a wavelength and amount effective to cause an alteration in the microbiome of the subject.

One embodiment provides a method of treating or preventing a cardiovascular condition in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

In one embodiment, the cardiovascular condition is atherosclerosis.

Typically, the light is administered to the subject at one or more locations on the subject which promote an alteration in the microbiome. In one embodiment, the light is administered to the abdomen of the subject.

One aspect provides a method of treating or preventing metabolic syndrome in a subject, comprising applying photobiomodulation therapy to the subject.

One aspect provides a method of reducing body weight and/or reducing cholesterol levels and/or reducing LDL levels and/or reducing serum triglyceride levels in a subject, comprising applying photobiomodulation therapy to the subject.

In one embodiment, the photobiomodulation therapy is administered to the abdomen of the subject.

As described in the Examples, the inventor has treated a mouse model of Parkinson's disease by applying light in the infrared wavelength to the abdomen of the mice. Application of the light to the abdomen of a number of mice resulted in an alteration of the microbiome of the mice, and an improvement in mobility of some of the mice. The inventors therefore reason that altering the microbiome using light in the infrared wavelength may result in improved mobility in neurological conditions such as Parkinson's disease.

As also described in the Examples, inventor has found that applying low level laser light in the infrared wavelength to the abdomen of humans suffering from Parkinson's disease resulted in an increase in the proportion of beneficial bacterial in their microbiome, and an improvement in sleep, balance, sense of smell, walking speed, cognitive ability, fine motor control, and outlook.

One aspect provides a method of treating or preventing a neurological condition in a subject, comprising administering to the subject light at a wavelength and amount effective to cause an alteration in the microbiome of the subject to increase the proportion of beneficial bacteria. Typically, the administering of light is photobiomodulation therapy.

One aspect provides a method of treating or preventing a neurological condition in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm). In one embodiment, the light is administered to the abdomen of the subject.

In one embodiment, the neurological condition is selected from the group consisting of Parkinson's disease and Alzheimer's disease. In one embodiment, the neurological condition is Parkinson's disease. In one embodiment, the neurological condition is Alzheimer's disease.

One aspect provides a method of treating or preventing Parkinson's disease in a subject, comprising treating the subject with light, typically photobiomodulation therapy (PBMT), at a wavelength and amount effective to cause an alteration in the microbiome of the subject.

One aspect provides a method of treating or preventing Parkinson's disease in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm). In one embodiment, the light is administered to the abdomen of the subject.

In one aspect, there is provided a method of increasing mobility of a subject suffering from Parkinson's disease, comprising treating the subject with light, typically photobiomodulation therapy (PBMT), at a wavelength and amount effective to cause an alteration in the microbiome of the subject.

One aspect provides a method of increasing mobility of a subject suffering from Parkinson's disease, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm). In one embodiment, the light is administered to the abdomen of the subject.

One aspect provides a method of treating a neurological condition in a subject, such as Parkinson's disease, comprising applying photobiomodulation therapy to the abdomen of the subject.

In one embodiment, the alteration in the microbiome comprises an increase in the proportion of at least one beneficial bacterial phylum, genus or species in the microbiome of the subject. In some embodiments in which the subject is a dog, rat or mouse, the beneficial bacterial phylum, genus or species may be Allobaculum sp.

In various embodiments in which the subject is a human, the beneficial bacterial phylum, genus or species may comprise one or more bacterial genus or species selected from the group consisting of:
(a) Akkermansia sp., Bifidobacterium sp., Lachnospira sp., Alistipes sp., Bacteroides sp., Coprococcus sp., Viellonella sp., Faecalibacterium sp., Roseburia sp., Dialister sp., Sutterella sp., Methanobrevibacter sp., and RF39;
(b) Lactobacillus sp., Bifidobacteria sp., Akkermansia sp., Faecalibacterium sp., Roseburia sp. and Bacteroides sp.
(c) Akkermansia sp., Lachnospira sp., Alistipes sp., Viellonella sp., Faecalibacterium sp., Roseburia sp., Dialister sp., Sutterella sp., Parabacteroides sp., and Prevotella sp.;
(d) Akkermansia sp., Bifidobacterium sp., Lachnospira sp., Alistipes sp., Coprococcus sp., Viellonella sp., Faecalibacterium sp., Roseburia sp., Dialister sp., Sutterella sp., Methanobrevibacter sp., and RF39; or
(e) Akkermansia sp., Bifidobacterium sp., Faecalibacterium sp., and Roseburia sp.;
(f) Akkermansia mucinophila, Bifidobacterium spp., Faecalibacterium prausnitzii and Roseburia spp.;
(g) Akkermansia mucinophila;
(h) Faecalibacterium prausnitzii;
(i) Bifidobacterium spp.;
(j) Roseburia sp.;
(k) Agathobacter sp.;
(l) Eubacterium sp.; or
(m) Ruminococcus sp.

In one embodiment, the beneficial bacteria are selected from the group consisting of Akkermansia mucinophila, Bifidobacterium longum., Faecalibacterium prausnitzii. and Roseburia inulinivorans.

In one embodiment, the beneficial bacterial phylum, genus or species is Akkermansia mucinophila.

In one embodiment, the alteration in the microbiome of the subject comprises decreasing the proportion of at least one unhealthy bacterial genus or species in the microbiome of the subject.

In one embodiment, the alteration in the microbiome of the subject comprises increasing the proportion of at least one beneficial bacterial phylum, genus or species, and decreasing the proportion of at least one unhealthy bacterial phylum, genus or species, in the microbiome of the subject.

In one embodiment, the alteration in the microbiome comprises a decrease in the ratio of the proportion of bacteria of the phylum Firmicutes to the proportion of bacteria of the phylum Bacteroidetes in the microbiome. In various embodiments, the decrease in the ratio of the proportion of bacteria of the phylum Firmicutes to the proportion of bacteria of the phylum Bacteroidetes is a decrease of at least 10%, 20%, 30%, 40%, 50%, 60% or 70% in the ratio.

One aspect provides a method of reducing the effects of ageing in a subject, comprising treating the subject with light, typically with photobiomodulation therapy (PBMT), at a wavelength and amount effective to cause an alteration in the microbiome of the subject.

One embodiment provides a method of reducing the effects of ageing in a subject, comprising administering to the subject an effective amount of light at a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm).

In one embodiment, the light is administered to the abdomen of the subject.

One aspect provides a method of reducing the effects of ageing in a subject, comprising applying photobiomodulation therapy to the abdomen of the subject.

One aspect provides a method of increasing resilience in a subject, comprising treating the subject with light, typically with photobiomodulation therapy (PBMT), at a wavelength and amount effective to cause an alteration in the microbiome of the subject.

One aspect provides a method of increasing resilience in a subject, comprising administering to the subject an effective amount of light of a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 811 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range of from 895 nm to 910 nm. In one embodiment, the wavelength is 904 nm to 905 nm (e.g., 904 nm or 905 nm). In one embodiment, the light is administered to the abdomen of the subject.

One aspect provides a method of increasing resilience in a subject, comprising applying photobiomodulation therapy to the abdomen of the subject.

As used herein, resilience is the ability of the subject to recover from an adverse health event. Typically, a subject having an increase in resilience is able to recover from an adverse health event is shorter time than a subject that does not have an increase in resilience.

As used herein, “preventing” means preventing a condition from occurring in a cell or subject that may be at risk of having or developing the condition, but does not necessarily mean that condition will not eventually develop, or that a subject will not eventually develop a condition. Preventing includes delaying the onset of a condition in a cell or subject. As used herein, “treating” in the context of treating a condition means reducing the extent or severity of a condition in a cell or subject relative to the extent or severity that would have been observed without applying the method.

As used herein, the term “subject” refers to a mammal such as a human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Typically the mammal is a human or primate. More typically, the mammal is a human. Although the present invention is exemplified using a mouse model, this is not intended as a limitation on the application of the present invention to that species, and the invention may be applied to other species, in particular, humans.

As used herein, the expressions “treating the subject with light”, “administering to the subject light” and “applying light to the subject” have same meaning as “irradiating the subject with light” and can be used interchangeably.

Photobiomodulation refers to the use of light to alter and/or modulate biological activity. Photobiomodulation therapy (PBMT) refers to exposing a subject to photons from, for example, a low level laser or a light-emitting diode (LED). In one embodiment, the photobiomodulation therapy is Low Level Laser Therapy (LLLT) or treatment with light-emitting diode (LED). LLLT refers to exposing subjects to a low level laser. As used herein, “low level laser” (LLL) is a laser that emits photons at a power density which is not sufficient to cause thermal damage to tissue. As used herein, a light-emitting diode is a semiconductor light source which emits photons at a power density which is not sufficient to cause thermal damage to tissue. As used herein, “power density” is the amount of power delivered per unit area. Power density is typically expressed in Watts per square centimetre. The power density used in photobiomodulation is typically in the range of from 1 to 500 mW/cm², 20-400 mW/cm², 40-300 mW/cm², 50-200 mW/cm², 50-120 mW/cm², 50-110 mW/cm², 100 to 500 mW/cm², 200 to 400 mW/cm², 250 to 400 mW/cm², 250 to 350 mW/cm². The power density of a laser or LED may be adjusted by adjusting the power output of the laser or LED and/or the distance of the laser or LED from the patient.

As used herein, an effective amount of light is a quantity of light energy that is sufficient to cause a desired effect.

As used herein, “energy density” is calculated by multiplying the power density by the amount of time (in seconds) the subject is subjected to the light during photobiomodulation therapy. Energy density is expressed in Joules/cm². In various embodiments, the energy density administered to the subject may be in the range of from 1-20 Joules/cm², 5-15 Joules/cm², 7-12 Joules/cm², 9-11 Joules/cm², 1-10 Joules/cm², 2-9 Joules/cm², 3-8 Joules/cm², 3-7 Joules/cm², 3-6 Joules/cm², 4-6 Joules/cm² or 4-5 Joules/cm², per point of irradiation As used herein, a point of irradiation is a location on a subject where a single light source is directed. A single treatment can comprise multiple points of irradiation. In various embodiments, a single treatment of a subject comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or at least 36 points of irradiation. For example, the patient may be treated at 8 or 9 points of irradiation of 5-15 Joules/cm² each at various points in the body. A treatment may comprise irradiating the subject at multiple points of irradiation carried out separately and in succession from a single source of light, or from multiple sources of light; or may comprise irradiating the subject at multiple points of irradiation carried out simultaneously from multiple light sources.

In one embodiment, the total light energy administered per treatment (in one day) is in the range of from 1-2000 Joules, typically 5-2,000 Joules, more typically 5-1,500 joules, 5-1000 Joules, 5-900 Joules, 5-800 Joules, 5-700 Joules, 5-600 Joules, 5-500 Joules, 5-400 Joules, 5-300 Joules, 5-250 Joules, 5-200 Joules, 5-150 Joules, 5-100 Joules, 5-90 Joules, 5-80 Joules, 5-70 Joules, 5-90 Joules, 5-90 Joules, 10-1,500 joules, 10-1000 Joules, 10-900 Joules, 10-800 Joules, 10-700 Joules, 10-600 Joules, 10-500 Joules, 10-400 Joules, 10-300 Joules, 10-250 Joules, 10-200 Joules, 10-150 Joules, 10-100 Joules, 10-90 Joules, 10-80 Joules, 10-70 Joules, 10-90 Joules, 10-90 Joules, 20-1,500 joules, 20-1000 Joules, 20-900 Joules, 20-800 Joules, 20-700 Joules, 20-600 Joules, 20-500 Joules, 20-400 Joules, 20-300 Joules, 20-250 Joules, 20-200 Joules, 20-150 Joules, 20-100 Joules, 20-90 Joules, 20-80 Joules, 20-70 Joules, 30-90 Joules, 40-90 Joules, or 50-90 Joules.

A subject may be treated daily (including one or more times daily), weekly, or once per week, twice per week, three times per week, four times per week, five times per week or six times per week.

The light is typically administered by photobiomodulation therapy. Photobiomodulation therapy (PBMT) is typically administered by subjecting the subject to photons emitted from a low level laser or LED. Typically, the PBMT is administered by subjecting a portion of the skin surface of the subject to photons. The photons are administered at a wavelength that promotes alteration in the microbiome of the subject. As used herein, alteration of the microbiome refers to changes in the proportion of microorganisms in the microbiome. For example, an alteration in the microbiome may be an increase in the proportion of a particular phylum, genus, species or strain of bacteria in the microbiome, and/or a decrease in the proportion of a particular phylum, genus, species or strain of bacteria in the microbiome.

The inventor has found that the light which is effective at altering the microbiome, and treating inflammatory and/or metabolic conditions, such as neurological and cardiovascular conditions, is in the infrared wavelength, typically near-infrared wavelength. In various embodiments, the wavelength which causes an alteration in the microbiome is in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 811 nm to 1050 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range from 895 nm to 910 nm. In one embodiment, the wavelength is in the range from 904 nm to 905 nm (e.g., 904 nm or 905 nm). In one embodiment, the wavelength is 904 nm. In one embodiment, the wavelength is 905 nm. In one embodiment, the wavelength is 808 nm.

In some embodiments, the photons are pulsed. The photons may be pulsed at a frequency in the range of 1-10,000 Hz, 1-100 Hz, 1-75 Hz, 30-60 Hz, 100-500 Hz, 200-500 Hz, 200-300 Hz, 200-5,000 Hz, 500-5,000 Hz, 1,000-5,000 Hz, 1,000-10,000 Hz, 2,000-10,000 Hz, 3,000-10,000 Hz, 4,000-10,000 Hz, 4,000-9,000 Hz, 4,000-8,000 Hz, 4,000-7,000 Hz, 4,000-6,000 Hz, 5,000-10,000 Hz, 6,000-10,000 Hz, or 7,000-10,000 Hz.

In one embodiment, the light is superpulsed. Light which is superpulsed typically has a pulse duration of 100-200 nanoseconds. Typically, the frequency of the pulse is in the range of from 1,000-10,000 Hz. In one embodiment, the wavelength of superpulsed light is 904 nm-905 nm. Light sources which are superpulsed include gallium-arsenide diode lasers and indum-gallium-arsenide diode lasers.

The light (e.g., laser or LED) may be applied to any location on the subject which results in an alteration of the subject's microbiome. Typically, the light (e.g., laser or LED) is targeted to a site on the skin surface of the subject. Typically, the light is targeted to a site at the abdomen of the subject. However, it is envisaged that the light may be targeted to other locations in addition to, or alternatively to, the abdomen. In one embodiment, the light is targeted to the abdomen. In another embodiment, the light is targeted to the abdomen and another location.

In various embodiments, the light (e.g., laser or LED) is targeted to one or more of the following sites of the subject per treatment:
(a) Skin surface at the abdomen of the subject;
(b) Skin surface at the cranial occipital junction and the upper cervicogenic region of the neck.

It will be understood that the specific dose level, energy density, and frequency of dosage for any particular subject may be varied and will depend upon a variety of factors including the age, body weight, general health, sex, diet, of the subject undergoing therapy.

Also provided is a light emitting device, such as a low level laser device or LED device, when used for preventing or treating a metabolic condition and/or inflammatory condition (such as a neurological condition and/or a cardiovascular condition and/or metabolic syndrome) in a subject, wherein the device is arranged to administer light, typically infra-red light, to the patient at a wavelength and location which causes an alteration in the subjects microbiome.

In one embodiment, the device is configured to administer light at a wavelength in the infra-red, typically near infrared, spectrum. In one embodiment, the device is configured to administer light at a wavelength in the range of from 630 nm to 1200 nm, typically in the range of from 635 nm to 1200 nm, 650 nm to 1200 nm, 670 nm to 1200 nm, 700 nm to 1200 nm, 780 nm-1080 nm, 800 nm to 1080 nm, 800 nm to 1050 nm, 800 nm to 950 nm, 800 nm to 910 nm, 800 nm to 850 nm, 800 nm to 840 nm, 800 nm to 810 nm, 809-1000 nm, 810 nm to 1050 nm, 820 nm to 1050 nm, 830 nm to 1050 nm, 840 nm to 1050 nm, 850 nm to 1050 nm, 860 nm to 1050 nm, 870 nm to 1000 nm, 870 nm to 1050 nm, 880 nm to 1050 nm, 890 nm to 1050 nm, 809 nm to 1000 nm, 810 nm to 990 nm, 811 nm to 1050 nm, 820 nm to 950 nm, 830 nm to 940 nm, 840 nm to 930 nm, 850 nm to 920 nm, 860 nm to 920 nm, 870 nm to 920 nm, 880 nm to 920 nm, 890 nm to 915 nm, 890 nm to 950 nm, 895 nm to 910 nm, 900 nm to 905 nm, 900 nm to 910 nm, 901 nm to 909 nm, 901 mnm to 908 nm, 901 nm to 907 nm, 902 nm to 906 nm, 903 nm to 905 nm, or 904 nm to 905 nm. In one embodiment, the wavelength is in the range from 895 nm to 910 nm. In one embodiment, the wavelength is 904 nm to 905 nm (e.g., 904 nm or 905 nm). In one embodiment, the wavelength is 904 nm. In one embodiment, the wavelength is 905 nm. In one embodiment, the wavelength is 808 nm.

In one embodiment, the device is configured to administer light to an abdomen of a subject. For example, the device may be configured in the form of a belt or strap which can be fitted around the waist of a subject to administer light to the abdomen of the subject.

One aspect provides a light emitting device for altering the microbiome of a subject, comprising an element configured to be worn by the subject, the element comprising one or more light sources which are capable of emitting light at a wavelength which causes an alteration in the microbiome of the subject, the one or more light sources being arranged in or on the element such that when the element is worn by the subject, the one or more light sources are positioned to direct light to the abdomen of the subject.

In one embodiment, the element is an abdominal securing element. Typically, the abdominal securing element is a belt or strap.

In one embodiment, the one or more light sources is a panel of low level laser lights.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order to exemplify the nature of the present disclosure such that it may be more clearly understood, the following non-limiting examples are provided.

### EXAMPLES

Photobiomodulation therapy (PBMT) has the potential to treat many of the diseases that are influenced by the microbiome, such as metabolic syndrome and its associated diseases, cardiovascular disease and neurological disorders (neuroinflammation) such as Parkinson's and Alzheimer's diseases.

### Example 1

We have carried out an experiment using healthy mice, where 4 mice per group were treated with various wavelengths of low-level laser as a pre-conditioning treatment against the symptoms of MPTP (1-methyl-4-phenyl-1,2,3,6-tetrahydropyridine) toxicity (Parkinson's disease model). Mice were treated with MPTP as described in Reinhart et al. Neuroscience Research 117: 42-47). In this experiment, the laser was an Irradia MID-LITE model 8080 with a wavelength of 808 nm, obtained from Spectro Analytic Irradia AB. Mice were treated by gently pressing the laser lens to the shaved abdomen. The laser spot size was 0.8 cm², with a 40° divergence of the laser beam, a pulse frequency of 250 Hz, a power density of 103.75 mW, a duty cycle of 11.25% and a total energy of 10 Joules. The microbiome was tested by extracting DNA from Faecal samples and subjecting the DNA to next generation sequencing (NGS). In total more than 10,000,000 sequences were compared across treatments.

Results showed that PBM applied to the mice in the infrared wavelength changed the microbiome composition, and in particular increased the proportion of one genus of mouse bacteria (Allobaculum sp.), which is known to be associated with a healthy microbiome and is reduced in unhealthy microbiomes (Bicknell B, Liebert A, Johnstone D, Kiat H. Photobiomodulation of the microbiome: implications for metabolic and inflammatory diseases. Lasers in medical science 2018:1-11).

A second experiment compared 808 nm and 904 nm low-level laser as a pre-conditioning treatment against the symptoms of MPTP toxicity, using healthy mice, 10 per group. Laser conditions for the 808 nm laser were as described above. For 904 nm, the laser spot size was 0.5 cm², with a 40° divergence of the laser beam, a pulse frequency of 250 Hz, an average power density of 60 mW (peak power 20 W), a duty cycle of 46% and a total energy density of 10 J/cm². The microbiome was tested by extracting DNA from Faecal samples and subjecting the DNA to next generation sequencing (NGS). In total 13,000,000 sequences have been compared between treatments. Results indicate that both infrared wavelengths can change the microbiome composition. However, 904 nm has more effect than 808 nm, changing microbiome composition more rapidly and affecting a greater number of bacterial species, with some species being increased and some being decreased. Species increased by laser treatment included species of Lactobacillaceae, Peicoccus, Ruminococcus and Prevotella among others, some of which are recognised as members of a healthy microbiome in mice. Species decreased by laser treatment include species of Burkholderales, Clostridium, Shuttleworthia and Nocardioides, some of which are considered to be unhealthy in the mouse microbiome.

### Example 2

In this experiment, a human subject was irradiated with laser light. Patient was a pre-diabetic volunteer who had received radiation therapy for breast cancer during the trial.

The laser was an Irradia MID-LITE model 904 with a wavelength of 904 nm, obtained from Spectro Analytic Irradia AB. The subject was treated by applying the laser lens to the abdomen. The laser spot size was 0.5 cm², with a 40° divergence of the laser beam, a pulse frequency of 750 Hz, a power density of 240 mW, a duty cycle of 46% and a total energy of 871 Joules. The microbiome was tested by extracting DNA from Faecal samples and subjecting the DNA to next generation sequencing (NGS). Faecal samples were self-collected by participants and held at −20° C. until processed. Genomic DNA was extracted using a commercial kit (Powerfecal® DNA extraction Kit from Qiagen). primers 514f (5′-GTGCAGAATTGCCCTATCC-3′) and 806r (5′-GACTACHVGGGTATCTAATCC-3′) were used to amplify the V4 hypervariable region of 16S rRNA of bacteria and archaea. Next generation sequencing was performed using the Illumina MiSeq@platform at the Australian Genomic Research Facility. Sequences were demultiplexed, quality trimmed and analysed using the Qiime2 program (version 2019-1 (https://docs.qiime2.org/2019.1/) using the DADA 2 plugin for quality control of sequences, the consensus method for removal of chimeras and the FastTree plugin to generate a phylogenetic tree with representative sequences. Taxonomy was assigned based on Greengenes (version 13_8; https://greengenes.secondgenome.com/) at 97% OUT, trained using a Naïve Bayes classifier. Diversity was calculated using at a rarefaction of 28,000 sequence sampling depth using Faith Phylogenetic Diversity, Evenness, Bray-Curtis and unweighted unifrac statistics.

Results of bacteria showing the most significant differences after treatment are shown in table 1.

TABLE 1

| | | Pre laser | Post laser |
|---|---|---|---|
| | Associated with | % of total | % of total |
| Bacteria associated with | | | |
| gut health | | | |
| Akkermansia mucinophila | Inversely correlated | undetected | 19.7 |
| | with obesity | | |
| Bifidobacterium sp | Short drain fatty | 4.7 | 13.2 |
| | acid production | | |
| Faecalibacterium sp | Short chain fatty | 0.5 | 5.8 |
| | acid production | | |
| Roseburia faecis and | Butyrate production | 5.0 | 9.9 |
| unidentified Roseburia sp. | Inversely correlate | | |
| | IBD | | |
| Bacteria associated with | | | |
| girt dysbiosis | | | |
| Ruminococcus lactaris | Chron's disease; | 4.2 | 1.6 |
| | rheumatoid arthritis | | |
| Collinsella tercoris | High blood | 2.9 | 0.4 |
| | triglycerides | | |
| Dorea longicatena | Insulin resistance | | |
| Holdmania | High blood | 0.32 | 0.11 |
| | triglycerides; | | |
| | glucose metabolism | | |
| Clostridium symbiosum | Pathogen | 0.38 | 0.21 |
| Eggerthella | Potential pathogen | 0.25 | 0.06 |

There are a number of bacterial groups that increase after laser treatment; these are bacteria that are associated with a healthy microbiome. The largest (and only significant) increase is for Akkermansia muciniphila which is associated with mucosal integrity, healthy mucous and short chain fatty acid production. Other good bacteria were Bifidobacterium sp., Faecalibacterium sp. and Roseburia faecis. There were a number of bacteria associated with gut dysbiosis or microbiome dysfunction that decreased after laser treatment. In this regard, there was a decrease in the proportion of Collinsella tercoris, Dorea longicatena, Clostridium symbiosum, and Eggerthella.

There was also a change in the Firmicutes: Bacteroidetes ratio which may be associated with body mass index and microbiome health. In this regard, the ratio went from 7.75 pre-PBMT to 3.88 post photobiomodulation therapy (PBMT) with a higher ratio believed to be associated with poorer microbiome health.

Results showed that PBMt applied to mice and human subjects in the infrared wavelength changed the microbiome composition, and in particular increased the proportion of beneficial bacteria in both mice and humans.

### Example 3

Five other human subjects with pre-metabolic syndrome were treated with the PBMT protocol to the abdomen.

### Treatment Regime:

Subjects were treated with 904 nm laser (4 diode cluster probe), 30 mW power density; 7.2 joules on 9 points around the abdomen—total joules=64.8; 3 times per week for between 5 and 12 weeks.

Single faecal samples were taken prior to and after photobiomodulation therapy (PBMt). Faecal samples were analysed in the same manner as in Example 2.

### Microbiome Results:

There were a number of bacterial genera associated with good gut health that were shown to increase after laser treatment (Table 2) and there were a number of bacterial genera associated with gut dysbiosis or microbiome dysfunction that decreased after laser treatment (Table 3). There were also bacterial genera of unknown function that either increased or decreased after PBMT. Some gut health bacterial genera decreased in some subjects and some dysbiosis genera increased in a small number of cases, but the general trend was overwhelmingly positive.

There was a decrease (improvement) in the Firmicutes: Bacteroidetes ratio in 3 of 6 subjects.

TABLE 2

| | Substantial | | Substantial | |
|---|---|---|---|---|
| | *Increase | | decrease | |
| | (n = 6) | | (n = 6) | |
| Genus | Subjects | Largest** | Subjects | Change |
| Associated with | showing | change in the | showing | in the |
| good gut health | increase | microbiome | decrease | microbiome |
| Methanobrevibacter | 1 | 0% → 0.53% | 0 | Undetected |
| Bifidobacterium | 4 | 1.4% → 12.9% | 0 | |
| Lachnospira | 3 | 0.13% → 1.6% | 2 | 0.8% → 0.1% |
| Alistipes | 2 | 0.003% → 0.02% | 0 | |
| Coprococcus | 4 | 1.0% → 4.9% | 0 | |
| Roseburia | 3 | 5.0% → 9.9% | 0 | |
| Faecalibacterium | 5 | 1.7% → 19.0% | 0 | |
| Dialister | 1 | 0.015% → 2.0% | 1 | 0.08% → 0.02% |
| Veillonella | 4 | 0.009% → 0.24% | 0 | |
| RF39 | 3 | 0.05% → 0.22% | 0 | |
| Akkermansia | 4 | 0.005% → 19.3% | 1 | 1.0% → 0.001% |
| Sutterella | 5 | 0.027 → 0.46% | 1 | 0.27% → 0.008% |

*an increase of at least 3-fold in % contribution to the microbiome
**the larges increase in the genus seen of the 6 subjects

TABLE 3

| | Substantial | | Substantial | |
|---|---|---|---|---|
| | decrease | | increase | |
| Genus | (n = 6) | | (n = 6) | |
| Associated | | Largest | Subjects | Change |
| with gut | | change in the | showing | in the |
| dysbiosis | | microbiome | increase | microbiome |
| Dorea¹ | 5 | 0.44% → 0.09% | 0 | |
| Boautia² | 5 | 19% → 5.8% | 0 | |
| Bilophila³ | 3 | 1.1% → 0.1% | 1 | 0.02% → 0.1% |
| Trabulsiella⁴ | 2 | 1.6% → 0.003% | 0 | |
| Ruminococcus⁵ | 4 | 2.7% → 0.4% | 1 | 0.02% → 1.0% |
| Turicibacter⁶ | 3 | 0.35% → 0.01% | 1 | 0.22% → 0.75% |
| Eggerthella⁷ | 3 | 1.9% → 0.04% | 1 | 0 → 0.005% |

*an increase of at least 3-fold in % contribution to the microbiome
**the largest increase in the genus seen of the 6 subjects
¹associated with IBS
²associated with obesity
³associated with increased gut inflammation
⁴associated with antibiotic gut disturbance
⁵associated with atrial fibrillation
⁶associated with IBD
⁷potential pathogen

### Example 4

Treatment of three human subjects suffering from high cholesterol.

### Treatment Regime:

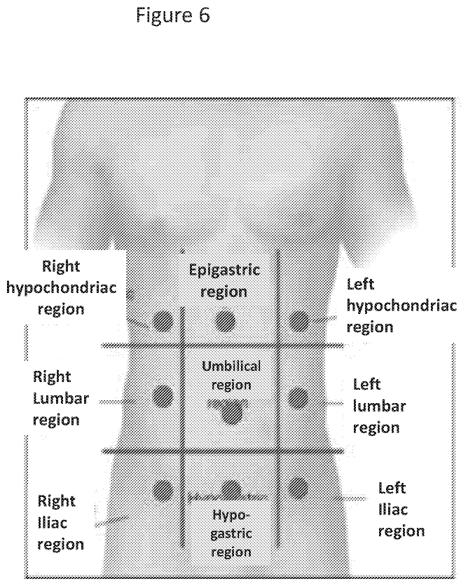

Patient was treated with 904 nm laser (4 diode cluster probe), 30 mW power density; 7.2 joules on 9 points around the abdomen as shown in FIG. 6—total joules=64.8; 3 times per week. Continued for 12 weeks. Exercise regime unchanged during PBMt trial. No dietary change.

Faecal samples were taken prior to and after PBMt. Faecal samples were analysed in the same manner as in Example 2.

### Results:

All subjects showed an improvement in cholesterol, LDL and triglycerides (2 of 3) (Table 4). Subject 1 lost 5.2 kg, subject 3 lost 3.2 kg over the treatment period.

TABLE 4

Lipid profile

| Subject 1 | O4/18 | 12/18 | 02/19 | post |
|---|---|---|---|---|
| cholesterol | 6.5 | 6.2 | 6.3 | 5.5 |
| LDL | 4.6 | 4.3 | 4.5 | 3.9 |
| triglycerides | 1.4 | 1.4 | 1.2 | 0.9 |

| Subject 2 | 10/18 | 02/19 | 06/19 | post |
|---|---|---|---|---|
| cholesterol | 7.1 | 6.6 | 7.7 | 5.2 |
| LDL | 4.8 | 4.3 | 5.3 | 2.7 |
| triglycerides | 0.9 | 0.8 | 0.8 | 0.7 |

| Subject 3 | 04/17 | 01/19 | post |
|---|---|---|---|
| cholesterol | 6.5 | 7.1 | 6.7 |
| LDL | 5.3 | 5.1 | 4.4 |
| triglycerides | 0.7 | 0.7 | 0.9 |

### Example 5

Subjects were suffering from Parkinson's disease and were divided into two groups, A and B. Group A were treated using photobiomodulation to the abdomen, neck and head for 12 weeks with a tapering of treatment (3 times per week for 4 weeks, 2 per week for 4 weeks, 1 per week for 4 weeks). Group B had the same treatment but waited 12 weeks before beginning. Before and after treatment, subjects were assessed for sleep (by self-report sleep-scale questionnaire), balance (tandem balance test with eyes closed), walking (speed test over 6 metres), timed up-and-go (time to stand from sitting in a chair, walk 3 metres and return to chair), and smell (subject-assessed sense of smell).

### Treatment Regime:

904 nm laser (4 diode cluster probe), 30 mW power density; 3.6 joules on 8 points around the abdomen—total joules=28.8; 7.2 joules to C1/C2; 20 minutes Vielight helmet and nasal LED.

Faecal samples were collected and the microbiome tested
Group A: immediately before PBMt, after 3 weeks of PBMt, after 12 weeks of PBMt.
Group B: 12 weeks before PBMt; immediately before PBMt, after 3 weeks of PBMt, after 12 weeks of PBMt
   Faecal samples were analysed in the same manner as in Example 2.

### Group A Results:

The changes in the microbiome was highly individual, as is the nature of Parkinson's disease. A number of subjects showed an increase in a number of bacteria recognised as being associated with a healthy microbiome, including Lachnospira, Dialister, Alistipes, Akkermansia and Sutterella (Table 5). A number of subjects also showed an increase in a number of bacteria that are normally depleted in Parkinson's patients, including Prevotella, Parabacteroides, Coprococcus and Roseburia. These are also associated with a healthy microbiome. A number of patients showed a decrease in numbers of bacteria that are often over-represented in Parkinson's patients, such as Christensenellaceae, Escherichia and Ruminococcus. These are often associated with dysbiosis. Interestingly, some bacteria that are over-represented in Parkinson's patients were increased in the subjects, including Akkermansia and Oscillospora (Table 5). These bacteria are often associated with a healthy microbiome. Also, some bacteria often depleted in Parkinson's patients were decreased during treatment in some of the subjects, including Blautia and Dorea, which are often associated with gut dysbiosis.

Additionally, the increase in beneficial bacteria observed was correlated with a positive symptomatic responses to the treatment. In this regard, a positive response to treatment was better sleep, better balance, improved sense of smell, reduced time taken to walk starting from a sitting position (“timed up-and-go”), increased walking speed, possible increased cognitive ability, increased fine motor control, improved outlook.

TABLE 5

| | No. of subject | No. of subjects | No. of subjects |
|---|---|---|---|
| | showing an | showing a | showing no |
| Genus | increase | decrease | change |
| Over-represented in | | | |
| Parkinson's | | | |
| microbiomes | | | |
| Bifidobacterium | 1 | 10 | 1 |
| Lactobacillus | 3 | 3 | 6 |
| Christensenellaceae | 3 | 5 | 4 |
| Ruminococcus | 2 | 4 | 6 |
| Escherichia/Shigella | 2 | 6 | 4 |
| Akkermansia | 7 | 1 | 4 |
| Oscillospora | 5 | 2 | 5 |
| Under-represented | | | |
| in Parkinson's | | | |
| microbiomes | | | |
| Parabacteroides | 7 | 1 | 4 |
| Prevotella | 8 | 2 | 2 |
| Coprococcus | 5 | 0 | 7 |
| Faecalibacterium | 2 | 3 | 7 |
| Roseburia | 4 | 4 | 4 |
| Dorea | 2 | 8 | 2 |
| Blautia | 1 | 6 | 5 |
| Associated with a | | | |
| healthy microbiome | | | |
| Dialister sp. | 7 | 1 | 4 |
| Lachnospira | 5 | 2 | 5 |
| Alistipes | 5 | 3 | 4 |
| Veillonella | 4 | 2 | 6 |
| Sutterella | 8 | 2 | 2 |
| Parabacteroides | 5 | 1 | 6 |

The results show that, following photobiomodulation therapy, there was an improvement in the symptoms of subjects suffering from Parkinson's disease (i.e., an improvement in sleep, balance, sense of smell, walking speed, cognitive ability, fine motor control, and outlook), which correlated with an increase in the proportion of beneficial bacteria in the subject's microbiome.

### Example 6

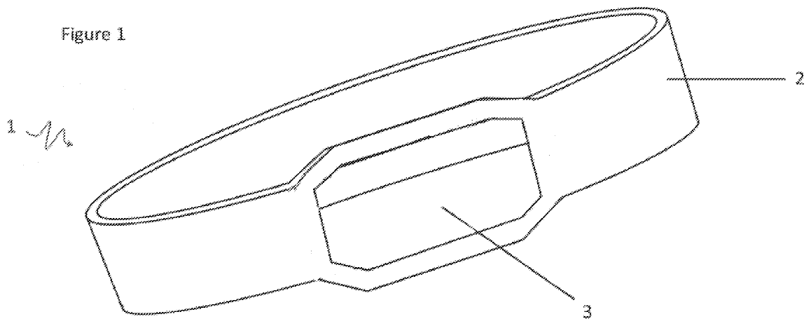

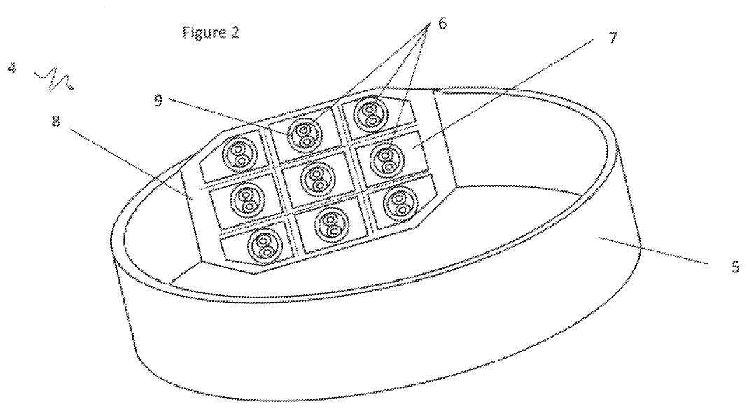

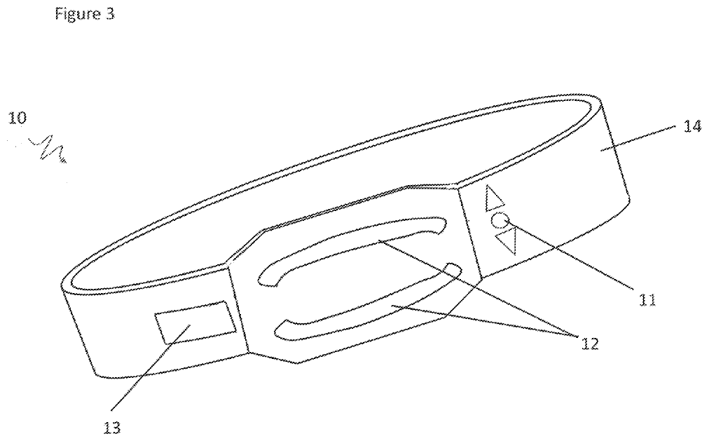

FIGS. 1 to 5 are an embodiment of a light emitting device for use in the method described herein. The device shown in FIGS. 1-5 is in the form of a belt for wearing around the abdomen of a subject. The belt comprises an inner layer (1, FIG. 1), a middle layer (4, FIG. 2) and an outer layer (10, FIG. 3). The inner layer (1) comprises a belt structure (2) formed from an elastomeric material, such as neoprene rubber, and comprises a window (3) which permits a light source placed at the window (from the middle layer) to irradiate the abdomen of the wearer. The middle layer (FIG. 2) comprises a belt structure (5), which fits over the belt structure of the inner layer (2), and a light emitting pad (8) which comprises an array of laser lights (6) in laser light assemblies (9). The belt structure of the middle layer (5) overlays the belt of the inner layer (2), such that the light emitting pad (8) is located at the window (3) of the inner layer (1) so that when the belt is worn, the lens of the laser light assemblies (9) are pressed directly against the abdomen of the wearer. The outer layer (10, FIG. 3) comprises a belt structure (14) which fits over the middle layer belt (5). The outer layer comprises an inflatable portion which can be inflated to cause pressure to be applied to the light emitting pad (8) of the middle layer so that it is pushed against the abdomen of the wearer. The outer layer comprises controls (11) that are operably connected to the laser lights (6) for controlling the output of the laser lights. The outer layer also comprises an LED display (12) operably connected to the controls and which indicates which laser lights (6) are operating. The outer lay also comprises a battery (13) operably connected to the laser lights.

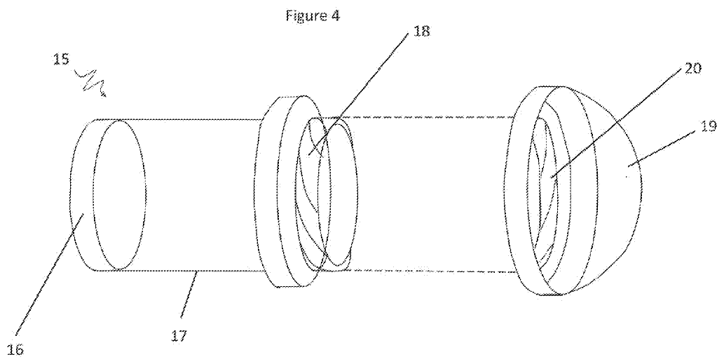

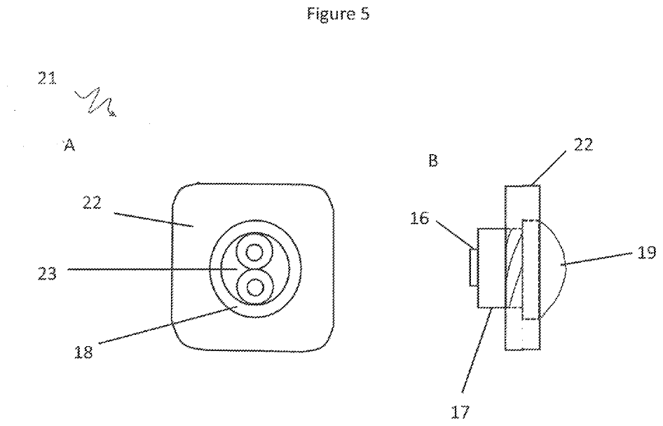

The array of laser lights comprises a panel of Class 1 Gallium Arsenide Laser lights housed in individual rigid plastic compartments embedded in a slightly flexible support. An example of an embodiment of the housing for each laser light assembly is shown in FIGS. 4 and 5. Each housing comprises a container (17) for receiving the laser light, with a conductive plate (16) for attachment of wires to connect each laser light to a power source. Each housing further comprises a threaded portion (18) for receiving the threaded portion of a lens (20). Each housing further comprises a heat sink to ensure that heat is not transferred to the wearer. The lens (19) comprises a transparent lens which is domed to increase special distribution of the laser to spread light evenly across the abdomen, and for comfort when placed against the abdomen of the user. The dome further comprises a threaded portion (20) for screwing the lens onto the body of the laser housing. The housing support around and between each laser light assembly is covered with a neoprene or fabric (21) for comfort when the light pad is pressed against the abdomen of the user.

A typical laser light produces a light having a wavelength of 904 nm+/−3 nm, with a laser bandwidth of +/−2 nm, an average power output of 10 mW+/−10%, peak power of 20,000 mW (per diode), a laser pulse duration of 60 ns, a laser pulse repetition rate of 50 Hz, and a beam area of 0.1 cm².

Output from the laser lights is controlled from the panel (11) on the outer layer of the belt.

It will be understood to persons skilled in the art of the invention that many modifications may be made without departing from the spirit and scope of the disclosure.

1. A method of preventing or treating a metabolic condition and/or an inflammatory condition in a subject, comprising treating the subject with light at a wavelength and amount effective to cause an alteration in the microbiome of the subject.

2. The method of claim 1, wherein the inflammatory condition is a neurological condition

3. The method of claim 2, wherein the neurological condition is Parkinson's disease.

4. A method of preventing or treating Parkinson's disease in a subject, comprising treating the subject with light at a wavelength and amount effective to cause an alteration in the microbiome of the subject.

5. The method of claim 1, wherein the metabolic condition is selected from the group consisting of type 2 diabetes, high blood pressure, high cholesterol levels, obesity, high LDL, and high serum triglycerides.

6. A method of reducing body weight in a subject, comprising treating the subject with light at a wavelength and amount effective to cause an alteration in the microbiome of the subject.

7. A method of reducing cholesterol levels and/or LDL levels and/or serum triglyceride levels in a subject, comprising treating the subject with light at a wavelength and amount effective to cause an alteration in the microbiome of the subject.

8. The method of any one of claims 1 to 7, wherein wavelength which causes an alteration in the microbiome of the subject is a wavelength in the range of from 809 nm to 1050 nm.

9. A method for altering the microbiome of a subject, comprising treating the subject with an effective amount of light at a wavelength in the range of from 809 nm to 1050 nm.

10. The method of claim 8 or 9, wherein the wavelength is in the range of from 820 nm to 1050 nm.

11. The method of claim 8, 9 or 10, wherein the wavelength is in the range of from 850 to 1050 nm.

12. The method of any one of claims 8 to 10, wherein the wavelength is in the range of from 895 nm to 910 nm.

13. The method of any one of claims 1 to 12, wherein treating the subject with light is photobiomodulation therapy.

14. The method of any one of claim 1 to 13, wherein the alteration in the microbiome is an increase in the abundance of one or more beneficial bacterial phylum, genus or species in the microbiome.

15. The method of any one of claim 1 to 14, wherein the alteration in the microbiome is a decrease in the ratio of the number of bacteria of the phylum Firmicutes to bacteria of the phylum Bacteroidetes.

16. The method of claim 14, wherein the one of more beneficial genus or species comprises a bacterial genus or species selected from the group consisting of Akkermansia sp., Bifidobacterium sp., Lachnospira sp., Alistipes sp., Bacteroides sp., Coprococcus sp., Viellonella sp., Faecalibacterium sp., Roseburia sp., Dialister sp., Sutterella sp.

17. A method of preventing or treating a metabolic condition and/or an inflammatory condition in a subject, comprising irradiating the subject with an effective amount of light at a wavelength in the range of from 809 nm to 1050 nm.

18. The method of claim 17, wherein the inflammatory condition is a neurological condition

19. The method of claim 18, wherein the neurological condition is Parkinson's disease.

20. A method of preventing or treating Parkinson's disease in a subject, comprising irradiating the subject with an effective amount of light at a wavelength in the range of from 809 nm to 1050 nm.

21. The method of claim 17, wherein the metabolic condition is selected from the group consisting of type 2 diabetes, high blood pressure, high cholesterol levels, obesity, high LDL, and high serum triglycerides.

22. A method of reducing body weight in a subject, comprising irradiating the subject with an effective amount of light at a wavelength in the range of from 809 nm to 1050 nm.

23. A method of reducing cholesterol levels and/or LDL levels and/or serum triglyceride levels in a subject, comprising irradiating the subject with an effective amount of light at a wavelength in the range of from 809 nm to 1050 nm.

24. The method of any one of claim 17 or 23, wherein the wavelength is in the range of from 820 nm to 1050 nm.

25. The method of any one of claims 17 to 24, wherein the wavelength is in the range of from 850 to 1050 nm.

26. The method of any one of claims 17 to 25, wherein the wavelength is in the range of from 895 nm to 910 nm.

27. The method of any one of claims 17 to 26, wherein treating the subject with light is photobiomodulation therapy.

28. The method of any one of claim 1 to 27, wherein treating the subject with light comprises applying the light to the abdomen of the subject.

29. A light emitting device when used for preventing or treating a metabolic condition and/or an inflammatory condition in a subject, wherein the device is arranged to administer light to the subject at a wavelength and location which causes an alteration in the microbiome of the subject.

30. The device of claim 29, wherein the inflammatory condition is a neurological condition

31. The device of claim 30, wherein the neurological condition is Parkinson's disease.

32. The device of claim 29, wherein the metabolic condition is selected from the group consisting of type 2 diabetes, high blood pressure, high cholesterol levels, obesity, high LDL, and high serum triglycerides.

33. The device of any one of claims 29 to 32, wherein wavelength which causes an alteration in the microbiome of the subject is a wavelength in the range of from 809 nm to 1050 nm.

34. The device of any one of claim 29 or 33, wherein the wavelength is in the range of from 820 nm to 1050 nm.

35. The device of any one of claim 29 or 34, wherein the wavelength is in the range of from 850 to 1050 nm.

36. The device of any one of claim 29 or 35, wherein the wavelength is in the range of from 895 nm to 910 nm.

37. The device of any one of claim 29 or 36, wherein the location is the abdomen.
